(19) United States
(12) Patent Application Publication
(10) Pub. No.: US 2012/0089103 A1
(43) Pub. Date: Apr. 12, 2012

(54) Medical Device For Healing Wounds and Skin Diseases

(76) Inventor: Ruben Tel-Ari, Tel Aviv (IL)

(21) Appl. No.: 12/899,630

(22) Filed: Oct. 7, 2010

(51) Int. Cl. A61M 35/00

(52) U.S. Cl. 604/290; 604/304; 604/293; 604/292; 604/305

(57) ABSTRACT

The present invention discloses a medical device having a disposable member for treating dermatological diseases and topical pathogeneses. The device comprises, inter alia, at least one inner layer at least partially made of a non-woven material, being in physical contact with at least a part of the derma having the dermatological diseases; at least one external impervious and flexible layer, at least partially made of material selected from a group consisting of silicone, rubber and polymer, enveloping the inner layer; and, source of hypertonic solution wetting the inner layer such that the inner layer is continuously immersed with the hypertonic solution and a constantly hypertonic environment around the derma is maintained. The inner layer is disposable and reversibly detachable to the enveloping external layer such that frequent displacement of the inner layer is possible, topical pathogeneses are being removed and dermatological diseases are being treated.

### CROSS REFERENCE TO RELATED APPLICATIONS

This application claims the benefit of priority from U.S. Provisional Application No. 61/042,793, which was filed on Apr. 7, 2008, and International Application No. PCT/IL2009/000295, which was filed on Mar. 16, 2009, of which the present application is a CIP.

### FIELD OF THE INVENTION

The present invention generally relates to the field of medical appliances. More particularly, the present invention relates to a medical device having a disposable member for treating the skin.

### BACKGROUND OF THE INVENTION

Lesions to the surface of the skin interrupt its homeostasis and may allow pathological events to destroy the well protected barrier of the skin. Liquids and/or Hyper osmotic materials have the ability to invert the pathological process by drawing liquids from the body towards the hyper osmotic area.

U.S. Pat. No. 4,622,035, which is incorporated herein by reference in its entirety, discloses a sock for the therapeutic or cosmetic treatment of the foot comprising a medicament supply network consisting of fingers, which continuously supplies a medicament to the absorbent lining and thence to the skin of the foot.

PCT Application No. WO9504511, which is incorporated herein by reference in its entirety, discloses a medical device comprising a skin patch with a target zone and an attached cover flap.

Although U.S. Pat. No. 4,622,035 and PCT Application No. WO9504511 disclose a medical device for continuously supplying medicament for treating the skin, none of them discloses means for providing a constant hypertonic environment around said infected skin portion.

Therefore there is still a long felt need for a medical device for treating a wounds and dermatological diseases whilst providing a disposable member in a constant hypertonic environment around the infected skin portion.

### SUMMARY OF THE INVENTION

It is thus one object of the invention to disclose a medical device having a disposable member for treating dermatological diseases and topical pathogeneses. The device comprises, inter alia, at least one inner layer at least partially made of a non-woven material, being in physical contact with at least a part of the derma having the dermatological diseases; at least one external impervious and flexible layer, at least partially made of material selected from a group consisting of silicone, rubber and polymer, enveloping the inner layer; and, source of hypertonic solution wetting the inner layer such that the inner layer is continuously immersed with the hypertonic solution and a constantly hypertonic environment around the derma is maintained; wherein the inner layer is disposable and reversibly detachable to the enveloping external layer such that frequent displacement of the inner layer is possible, topical pathogeneses are being removed and dermatological diseases are being treated.

Another object of the invention is to disclose the medical device as defied above, wherein the dermatological diseases and topical pathogeneses comprising means for immobilizing, useful for reversibly tightening the medical device to the derma such that leakage of the hypertonic solution is prevented.

Another object of the invention is to disclose the medical device as defied above, wherein the supplying means is adapted to provide flow-regulation of the hypertonic solution selected from a group consisting of continuous flow, intervals flow, pulsed flow, programmed flow, pre-programmed flow, flash, circulated or any combination thereof.

Another object of the invention is to disclose the medical device as defied above, wherein the device additionally comprises a thermoregulator for thermoregulating the temperature of the solution.

Another object of the invention is to disclose the medical device as defied above, wherein the inner layer comprising at least two layers: at least one inner carrier layer being in physical contact with the derma; and at least one middle layer for temporarily containing the hypertonic solution.

Another object of the invention is to disclose the medical device as defied above, wherein the hypertonic solution comprising at least one member of a group consisting of salt, sugar, honey, 50% dextrose, hypertonic saline, oils, normotonic solutions added with therapeutic aides or any combination thereof.

Another object of the invention is to disclose the medical device as defied above, wherein the hypertonic solution is selected from a group consisting of antiseptic solution, a lipophilic solution, an antibiotic solution, an antifungal solution, an antiviral solution or any combination thereof.

Another object of the invention is to disclose the medical device as defied above, wherein the hypertonic solution comprises at least one member of group consisting of air, oxygen or oxygen-rich liquid, H₂O₂ and peroxides.

Another object of the invention is to disclose the medical device as defied above, wherein the configuration of the medical device is selected from a group consisting of a sock-like configuration, glove-like configuration, strap, pre-rolled tube, head-like mask, underwear-like configuration, genital cover, genital wraps, diaper-like, pressure pads, orthopedic foot support.

Another object of the invention is to disclose the medical device as defied above, wherein source of hypertonic solution compromises at least one member of a group consisting of at least one pipe, a network of pipes and of at least one pressure cup for circulating the hypertonic solution within the medical covering.

Another object of the invention is to disclose the medical device as defied above, wherein the device further comprising purification means, selected from one or more members of a group consisting of beads, fibers, mesh, membrane and filter.

Another object of the invention is to disclose the medical device as defied above, wherein the purification means comprises one member of a group consisting of zinc oxide, black carbon, metal ions, hypochlorite salts, halogen salts, quaternary amine and salts thereof.

Another object of the invention is to disclose the medical device as defied above, wherein the purification means is located in one of the following: (i) within the at least one inner layer, or (ii) in fluid communication with the source of hypertonic solution.

Another object of the invention is to disclose the medical device as defied above, especially adapted for de-toxification of dermal surface.

Another object of the invention is to disclose a method for treating dermatological diseases and topical pathogeneses. The method comprises steps of: obtaining a device comprising at least one inner layer at least partially made of a non-woven material, being in physical contact with at least a part of the derma having the dermatological diseases; at least one external impervious and flexible layer, at least partially made of material selected from a group consisting of silicone, rubber and polymer, enveloping the inner layer; and a source of hypertonic solution wetting the inner layer such that the inner layer is continuously immersed with the hypertonic solution and a constantly hypertonic environment around the derma is maintained; introducing the device upon at least a part of the derma associated with the dermatological diseases; continuously setting at least one inner layer with the hypertonic solution such that a constant hypertonic environment around the derma is maintained; and frequently replacing the disposable and reversibly detachable at least one inner layer such that topical pathogeneses are removed and dermatological diseases are treated.

Another object of the invention is to disclose the method as defied above, wherein the method additionally comprises steps of providing flow-regulation of the hypertonic solution; and selecting the same from a group consisting of continuous flow, intervals flow, pulsed flow, programmed flow, pre-programmed flow, flash, circulated or any combination thereof.

Another object of the invention is to disclose the method as defied above, wherein the method additionally comprises step of thermoregulating the temperature of the solution.

Another object of the invention is to disclose the method as defied above, wherein the method additionally comprises step of selecting the dermatological diseases from a group consisting of skin ulcers, particularly diabetic skin ulcers, skin burns of grades 1, 2 or 3, frost bites, psoriasis, hypertrophic skin diseases, edemas, circulatory deficiencies, pressure sores, sport injuries, articulation traumas, decompression injuries.

Another object of the invention is to disclose the method as defied above, wherein the method additionally comprises steps of composing the hypertonic solution, and selecting the same from one or more member of a group consisting of: salt, sugar, honey, 50% dextrose, hypertonic saline, oils, normotonic solutions added with therapeutic aides or any combination thereof.

Another object of the invention is to disclose the method as defied above, wherein the method additionally comprises step of selecting the hypertonic solution from one or more member of a group consisting of antiseptic solution, a lipophilic solution, an antibiotic solution, an antifungal solution, an antiviral solution or any combination thereof.

Another object of the invention is to disclose the method as defied above, wherein the method additionally comprises steps of configuring the medical device in a configuration; and selecting the same from a group consisting of a sock-like configuration, glove-like configuration, strap, pre-rolled tube, head-like mask, genital cover, genital wraps, diaper-like, pressure pad, orthopedic foot support.

Another object of the invention is to disclose the method as defied above, wherein the method additionally comprises of regulating the flow of the hypertonic solution.

Another object of the invention is to disclose the method as defied above, wherein the method additionally comprises step of circulating the hypertonic solution whilst homogeneously distributing pressure within the medical device via coupling at least one pressure cup to the external layer.

Another object of the invention is to disclose the method as defied above, wherein the method additionally comprises step of providing the source of hypertonic solution to comprise at least one member of a group consisting of at least one pipe, a network of pipes and of at least one pressure cup for circulating the hypertonic solution within the medical covering.

Another object of the invention is to disclose the method as defied above, wherein the method additionally comprises steps of providing a means of fluid purification, and selecting the means of fluid purification from one or more members of a group consisting of beads, fibers, mesh, membrane and filter.

Another object of the invention is to disclose the method as defied above, additionally comprising step of de-toxificating the dermal surface.

Still another object of the invention is to disclose the method as defied above, wherein the method additionally comprises steps of providing a means of fluid purification, and selecting the means of fluid purification from member of a group consisting of zinc oxide, black carbon, metal ions, hypochlorite salts, halogen salts, quaternary amine and salts thereof.

The last object of the invention is to disclose a disposable member for treating dermatological diseases and topical pathogeneses. This replaceable member is characterized by (i) size and shape which fit derma to be treated, (ii) by a matrix at least partially made of non-woven medical useful for being continuously wet by a hypertonic solution; and (iii) by compatibility with at least one covering external layer facilitating the member to be constantly wet.

### BRIEF DESCRIPTION OF THE DRAWINGS

The subject matter regarded as the invention will become more clearly understood in light of the ensuing description of embodiments herein, given by way of example and for purposes of illustrative discussion of the present invention only, with reference to the accompanying drawings wherein:

FIG. 1 is an illustration of a medical device for healing wounds and skin diseases formed as a therapeutic sock, according to some embodiments of the invention;

FIG. 2 is an illustration of an external sock for covering an inner sock, according to some embodiments of the invention;

FIG. 3 is an illustration of a medical device for healing wounds and skin diseases formed as a body wrap, according to some embodiments of the invention;

FIG. 4 is an illustration of application and details of a medical device for healing wounds and skin diseases formed as a body wrap, according to some embodiments of the invention;

FIG. 5 is an illustration of a medical device for healing wounds and skin diseases formed as a healing glove, according to some embodiments of the invention;

FIG. 6 is an illustration of a medical device for healing wounds and skin diseases formed as a sock wrapping, according to some embodiments of the invention;

FIG. 7 is an illustration of a medical device for healing wounds and skin diseases formed as a sock wrapping and applied to the head of a user, according to some embodiments of the invention;

FIG. 8 is an illustration of a medical device for healing wounds and skin diseases formed as a head mask and applied to the head of a user, according to some embodiments of the invention;

FIG. 9A, 9B, 9C are illustrations of a medical device for healing wounds and skin diseases formed as pockets upon the inner side of an underwear, according to some embodiments of the invention;

FIG. 10 is a flowchart illustrating a method for healing wounds and skin diseases, according to some embodiments of the invention; and

FIGS. 11A-11M illustrate orthopedic foot support containing at least two water/air reservoirs for a better pressure scattering.

### DETAILED DESCRIPTIONS OF SOME EMBODIMENTS OF THE INVENTION

The following description is provided, alongside all chapters of the present invention, so as to enable any person skilled in the art to make use of the invention and sets forth the best modes contemplated by the inventor of carrying out this invention. Various modifications, however, will remain apparent to those skilled in the art, since the generic principles of the present invention have been defined specifically to provide a medical device having a disposable member useful for maintaining damaged skin in a therapeutic solution environment is disclosed.

The medical device can be used for mammals, pets (veterinary used) or for humans.

The principles and uses of the teachings of the present invention may be better understood with reference to the accompanying description, figures and examples. It is to be understood that the details set forth herein do not construe a limitation to an application of the invention. Furthermore, it is to be understood that the invention can be carried out or practiced in various ways and that the invention can be implemented in embodiments other than the ones outlined in the description below.

The term ‘hypertonic solution’ or ‘Hyperosmotic’ refers to any solution/liquid which has a higher concentration of solutes than the cells with which it is in contact, so that water is drawn out of the cells and into the solution by osmosis. The hypertonic solution is possibly provided useful as a human medicament and/or veterinary medicament as being a solution with effective therapeutic characteristics for humans and/or animals.

The term ‘additives’ refers hereinafter to either organic (natural occurring or synthesized materials) or inorganic compositions, in a fluid, gas or solid state, selected in a non-limiting manner form a group consisting, inter alia, of biocides, medicaments, narcotics, pain-relieving agents, heparin and heparin-like agents, anti-coagulants or coagulation factors, pharmaceuticals, binders, pigments, emulsifiers or soaps, de-emulsifiers, solvents, oils, plant extracts, essential oils, perfumes, sustain released drugs, markers, biomarkers, electrolytes, enzymes, hormones, proteins, vitamins, nutrients, or any combination thereof.

The term ‘carrier’ refers to any fluid that is characterized by being able to withdraw water from living cells, the fluid being in any appropriate form, including but not limited to liquids, solutions (whether water-miscible or water-immiscible), organic solvents, suspensions, dispersions, emulsions, fluid polymers, finely divided solids, nano-particles, micro-particles, powders, fine powders, gases, gels, aerosols, supercritical fluids, ionic liquids, surfactants, liposomes or any combination thereof.

According an embodiment of the invention, the term ‘salts’ refers to one or more compositions that are selected in a non-limiting manner from one or more compositions selected from a group consisting, inter alia, of one or more cations, such as sodium, potassium, magnesium or calcium; and one or more anions, such as chlorides, hydroxides, phosphates or ammonium; or any combination thereof.

The term ‘sugars’ refers to one or more of four chemical groupings of carbohydrates: monosaccharide, disaccharide, oligosaccharide, and polysaccharide. The term ‘honey’ refers to any natural occurring honey, or honey-like synthesized compositions, such as compositions comprise ingredients as follows e.g., fructose: about 38.0%, glucose: about 31.0%, sucrose: about 1.0%, water: about 17.0%, other sugars: about 9.0% (maltose, melezitose), ash: about 0.17% and additives: about 3.38% (weight percent).

The term ‘hydrogels’ refers to any composition adapted to comprise more than 98% water. It is one embodiment of the invention wherein cationic polymers are utilized, such as copolymers of vinylpyrrolidone, methacrylamide, and N-vinylimidazole. It is another embodiment of the invention wherein Poly ethylene glycol (PEGs) familyare utilized. The family comprises, inter alia, PEG, polyethylene oxides (PEOs) or polyoxyethylenes (POEs) etc.

The term ‘about’ refers hereinafter to ±20% of the defined measure.

The term ‘a plurality’ refers hereinafter to an integer number, equal or greater than 1.

The term ‘medical solution’ refers hereinafter to any used medical solution especially 50% dextrose, oils, saline, hypertonic solution saline 9%, gel, liquefied gel, Aloe-vera, emulsion (e.g., milk, goats' milk, casein, mixed emulsions), L-Carnitine, albumin, carnitine, honeydew, Propoli, propolis concentrated plants or flowers extracts, Dead sea water.

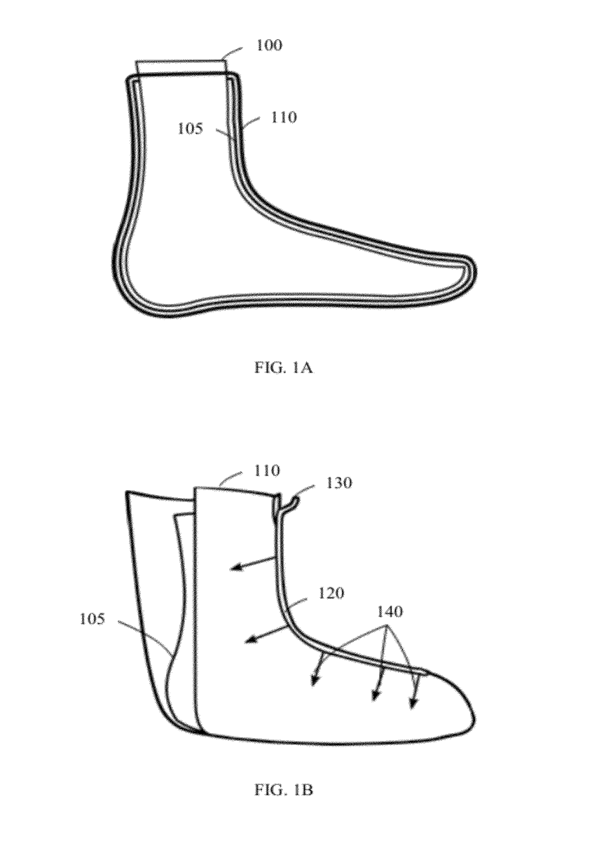

FIG. 1 is an illustration of a medical device for healing wounds and skin diseases formed as a therapeutic sock-like medical covering, according to one embodiments of the invention.

FIG. 1A illustrates a cross-section of the device and a patient's foot (100) temporarily accommodated within the same. Inner layer 105 is a disposable member, preferably made of non-woven materials, at least partially surrounding/enveloping the user's foot 100.

FIG. 1B illustrates an optional external impervious and flexible layer 110 at least partially enveloping/surrounding the disposable inner layer 105. The external layer 110 comprises at least one supplying means (namely tube 120) for delivering liquids into the external layer 110. Liquids flows into the external layer 110 through opening 130 and are allowed to flow 140 inside the external layer and soak the disposable inner layer 105. The tube 120 may be connected to a network of fluid-flowing pipes within the medical covering, facilitating continuous flow, intervals of pulsed flow or programmed flow of the hypertonic solution into the external layer 110 and/or inner layer 105.

The network of fluid-flowing pipes is possibly provided with a fluid connection with at least one fluid inlet/outlet 130 and/or with a plurality of conducting pipes (See 140) facilitating a flow of the solution within external layer 110, such that disposable inner layer 105 is effectively soaked, impregnated, immersed, doped or otherwise wet by the solution and patient's foot to be cured is accordingly wet by the hypertonic solution.

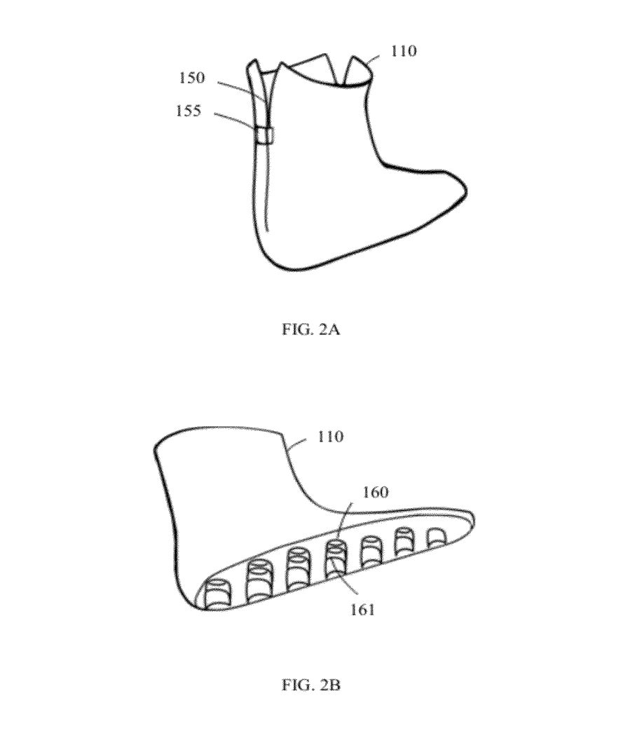

Reference is now made to FIG. 2A and FIG. 2B, illustrating in an out-of-scale manner an external sock-like member designed for covering an inner sock-like member according to an embodiment of the invention.

FIG. 2A illustrates the external layer 110 adapted to be in an either CLOSE or OPEN configurations. CLOSE configuration is provided in a non-limiting manner by a water-sealed zipper 155 provided, e.g., along the rear portion 150 of the device.

FIG. 2B illustrates pressure cups (full 160 and in cross section 161) provided on the inside of the external layer's 110 sole. The pressure cups are adapted to cause inner liquids to circulate in the sock.

Care should be taken to distinguish between pressures cups used as shock absorbers which are positioned on the outside of the shoe (usually on the outside of the sole) and the pressures cups that are provided by the present invention which are positioned internally to the shoe.

It should be emphasized that the pressure cups can be a part of the external layer—made of materials such as rubber etc. Alternatively they can be homogeneously spread within the medical cover to reduce pressure and to create a suction force to enable the liquid solution to flow around pressurized organs such as foot while walking.

According to another embodiment, the liquids (such as water, saline, oils et cetera) or the hypertonic solution is circulated within the cups.

According to another embodiment, the cups are sealed such that leakage of the liquids or the hypertonic solution from the cups is prevented.

According to another embodiment, the cups are open such that a full circulation of the liquids or the hypertonic solution from the cups to the patient's organ is enabled.

According to another embodiment, the cups are air filled.

The main advantage of such embodiments is the fact that there is a homogeneous distribution of pressures and better circulation of the liquids and/or the hypertonic solution around the organ.

It should be pointed out that the pressures cups can be randomly distributed or according to predetermined protocols.

Therefore, according to another embodiment of the present invention, pressure pads are provided. In which a plurality of pressure cups are either homogeneously or randomly spread. As described above, the pressure cups can be sealed or open. They can be filled with air or any liquid for better stress distribution (in case of air) or better circulation of the liquid.

According to another embodiment the pressure pads can be foot support (orthopedic use).

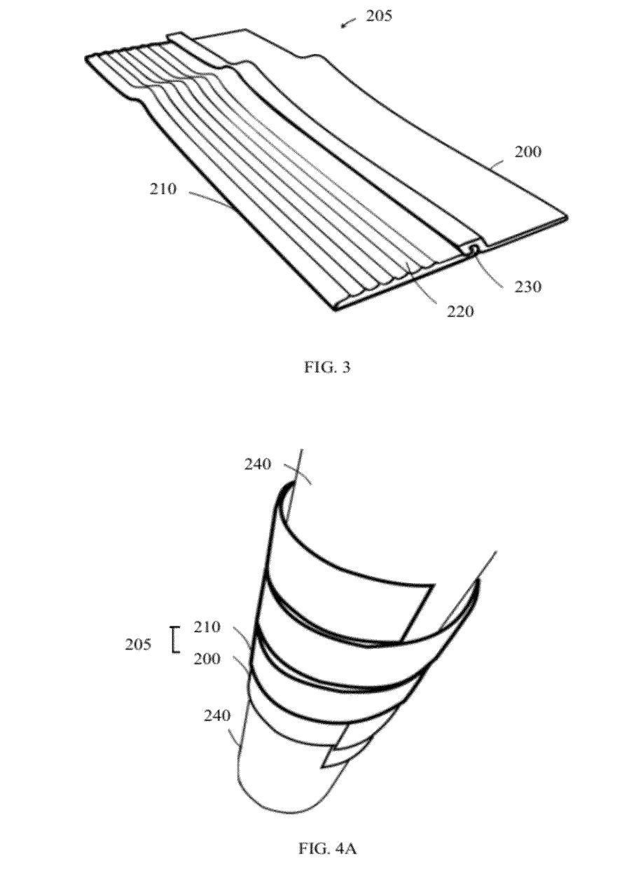

FIG. 3 is an illustration of a medical device for healing wounds and skin diseases formed as a body wrap, according to some embodiments of the invention. The body wrap strap 205 comprises of an inner layer 200 soaked, impregnated, doped or otherwise wet by a therapeutic solution, namely a hypertonic solution, connected with at least one joint 230 to an external layer 210 which is impervious and flexible. The external layer 210 comprises a plurality of channels 220 (e.g., a network of fluid-flowing pipes) running along the strap for directing the flow of the therapeutic solution, namely hypertonic solution. The channels 220 may comprise a network of fluid-flowing pipes running along the strap, adapted to facilitate the flow of the therapeutic solution.

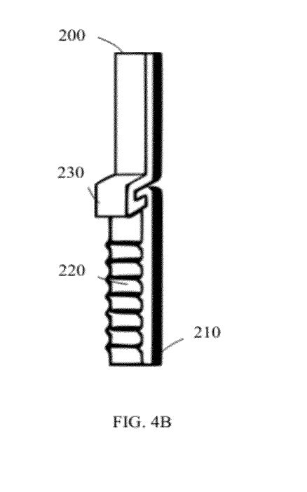

FIG. 4 is an illustration of application and details of a medical device for healing wounds and skin diseases formed as a body wrap, according to some embodiments of the invention.

FIG. 4A illustrates the application of the body wrap strap 205 winded around a patient's limb 240. The external layer 210 covers the internal layer 200 that was winded around the user's limb 240. FIG. 4B illustrates a perspective view of the connecting joint 230 provided between the external layer 210 and the internal layer 200. The external layer 210 comprises channels 220 running along the strap for directing the flow of the therapeutic solution, e.g., the hypertonic solution.

According to some embodiments of the invention, in the body wrap, the internal layer 200 and external layer 210 are substantially co-planar.

According to some embodiments of the invention, the external layer 210 and/or the internal layer 200 of the body wrap may comprise highly flexible pressure cups for facilitating the flow of the hypertonic solution, e.g., for treating pressure sores and the like.

It is in the scope of the invention wherein the internal 200 and external 210 layers are positioned in about a co-planar manner.

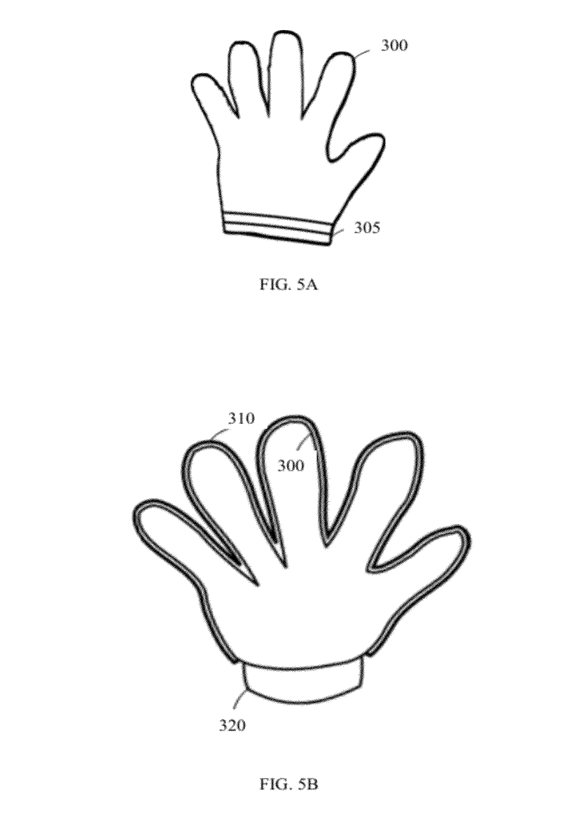

FIG. 5 is an illustration of a medical device for healing wounds and skin diseases formed as a healing glove-like medical covering, according to some embodiments of the invention. FIG. 5A illustrates a disposable inner layer 300 of the glove-like medical device provided with one or more immobilizing means, here stripes 305, for reversibly tightening the glove to a user's hand 320 and absorbing excess liquids from the glove; thus, leakage is prevented.

FIG. 5B illustrates the disposable inner 300 and outer 310 layers of the medical device glove. In this manner, the glove is worn on the user's hand 320 keeps the skin in constant contact with a therapeutic solution, namely the hypertonic solution.

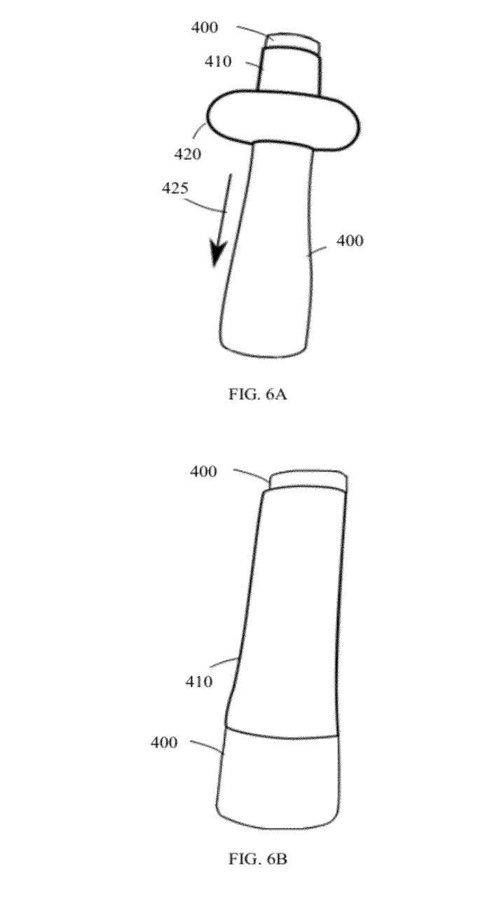

FIG. 6 is an illustration of a medical device for healing wounds and skin diseases formed as a sock-like wrapping, according to some embodiments of the invention. The sock wrapping 410 is designed as a pre-rolled tube adapted to be applied over a limb 400 or any other tubular organ of the patient. The sock wrapping 410 is applied by unrolling (see arrow 425, FIG. 6A) the pre-rolled part 420 of the tube until there is a full coverage 410 of the part of limb to be treated.

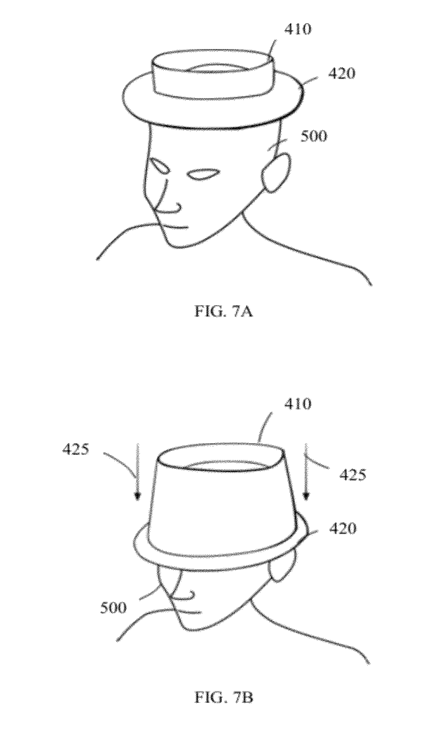

FIG. 7 is an illustration of a medical device for healing wounds and skin diseases formed as a sock (hat-like) wrapping and applied to the head of a user, according to some embodiments of the invention. The sock wrapping 410 formed as a pre-rolled tube applied to the head 500 of a user. The sock wrapping 410 is applied by unrolling 425 the pre-rolled part 420 of the tube until it fully covers 410 the part of head to be treated.

FIG. 8 is an illustration of a medical device for healing wounds and skin diseases formed as a head mask 600 and applied to the head of a user, according to some embodiments of the invention. The Head mask 600 is adapted to be enveloped at least a portion of the face and/or head of a patient.

Head mask 600 may be attached to the user's by closing an aperture 605 in mask 600. Mask 600 is made of non-woven fabric, and might include detachable parts for the eyes 610, nose 620, mouth 630 and ears 640. The detachable parts are designed by means of size and can be removed to set free the corresponding head parts that are to be left untreated. The rims of the detachable parts are optionally perforated for easy tearing. Removing the detachable parts leaves gaps for the ears 645 and gaps for the face 615.

It is emphasized that the mask is fluid tight at the circumference of the gaps left for the ears 645 and face 615.

According to some embodiments of the invention, the inner layer of the sock wrap 410 and/or mask 600 are made of non-woven material and soaked, impregnated, doped or otherwise wet by a therapeutic solution, and an external layer made of an impervious and flexible material such as silicone, rubber, polymer etc.

According to yet another embodiment of the invention, sock wrap 410 and mask 600 comprises (i) at least one inner layer, at least partially made of a non-woven material and soaked, impregnated, doped or otherwise wet by a hypertonic solution, and (ii) at least one external layer, made of e.g., an impervious and flexible material such as silicone, rubber, polymer etc.

According to yet another embodiment of the invention, the above mentioned types of medical device are potentially solely made of one or more external layer, keeping the skin beneath it in constant contact with a hypertonic solution, without utilizing an internal layer for this purpose.

According to yet another embodiment of the invention, the external layer is made of a multi-use silicone, rubber or polymer, and the inner layer is made of a disposable non-woven material. The external layer is possibly adapted to hold a reservoir of the hypertonic solution.

The external layer is possibly adapted to be warmed or cooled as part of the treatment. Hence, a cooler, heater of a combined temperature regulator is utilized to control the solution temperature. A Peltier cooler/heater is found useful.

According to yet another embodiment, the medical covering, as defined hereinabove, can be used as genital wraps.

According to yet another embodiment of the invention, the medical covering, as defined hereinabove, are especially adapted to treat skin ulcers, particularly diabetic skin ulcers, skin burns of grades 1, 2 or 3, frost bites, psoriasis, hypertrophic skin diseases, edemas, circulatory deficiencies, pressure sores, sport injuries, articulation traumas, decompression injuries.

According to yet another embodiment, the medical covering, as defined hereinabove, is especially constructed and used for de-toxification of dermal surface.

It is still in the scope of the invention to provide a method for treating topical- (e.g., dermal-) pathogeneses in mammals and especially humans, e.g., skin ulcers, particularly diabetic skin ulcers, skin burns of grades 1, 2 or 3, frost bites, psoriasis, hypertrophic skin diseases, edemas, circulatory deficiencies, pressure sores, sport injuries, articulation traumas, decompression injuries. The method comprises steps of (i) obtaining a medical device as defined in any of the above; (ii) soaking, impregnating, doping or otherwise wetting the body portion of be treating by the solution.

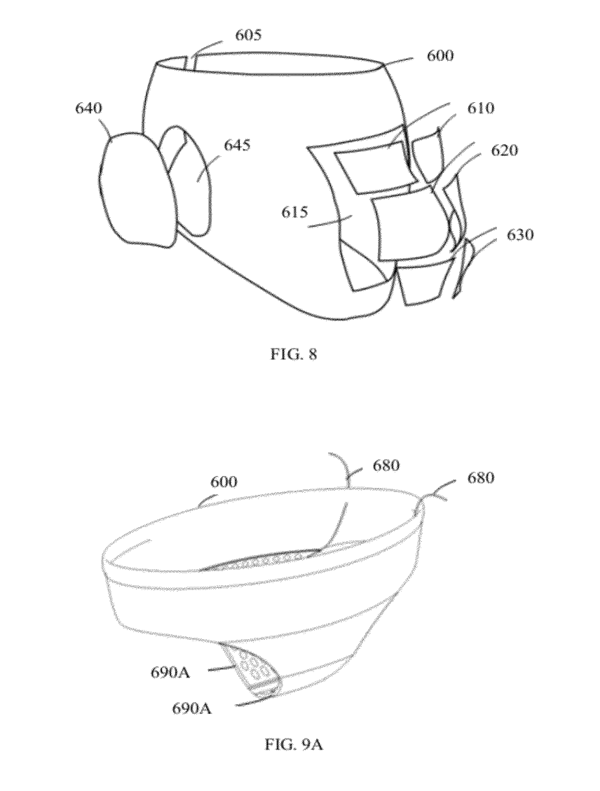

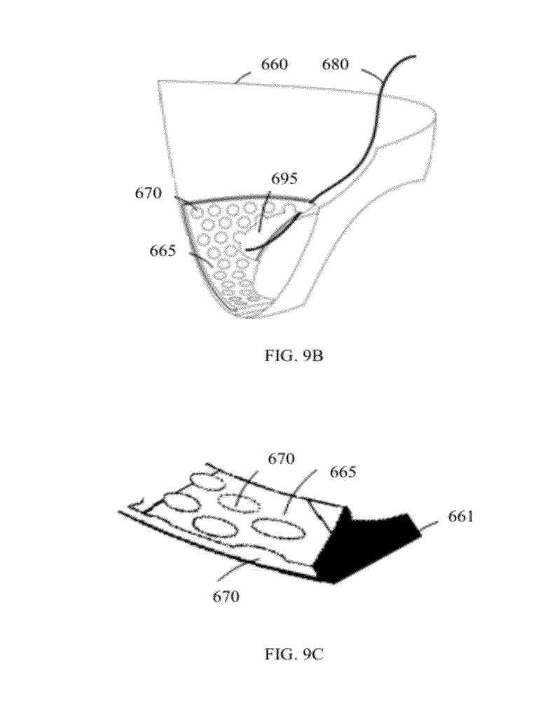

Reference is now made to FIGS. 9A, 9B, 9C are illustrations of a medical device for healing wounds and skin diseases formed as pockets 690 upon the inner side of an underwear 660, according to some embodiments of the invention. FIG. 9A is a perspective view, FIG. 9B a cross section along the front-rear plane, and FIG. 9C is an enlarged view of the medical covering. The medical device may be formed as a front pocket 690A and as a rear pocket 690B. Each pocket 690 comprises an internal fabric mesh 665 upon the underwear fabric 661. Between the internal fabric mesh 665 and the underwear fabric 661, a removable onetime non-woven layer 670 may be inserted and soaked with a therapeutic solution. A feeding line 680 connected to the removable and disposable onetime non-woven layer 670 may introduce the therapeutic solution to the removable onetime non-woven layer 670 (as illustrated by removing a part of the internal fabric mesh 665 in area 695), and drain excess solution. The underwear fabric 661 is impermeable and the rims of the pockets 690 prevent leakage.

According to some embodiments of the invention, the above mentioned types of medical device may be made of an external layer only, keeping the skin beneath it in constant contact with a therapeutic solution but without utilizing an internal layer for this purpose.

According to some embodiments of the invention, the therapeutic solution is a hypertonic solution of either a concentrated sugar solution, such as honey or 50% dextrose, or a solution of salts.

According to some embodiments of the invention, the therapeutic solution may be at least one of the following: a general antiseptic solution, a lipophilic solution, an antibiotic solution, an antifungal solution, an antiviral solution.

According to some embodiments of the invention, the solution in the inner layer may comprise air, oxygen or an oxygen-rich liquid (such as H₂O₂ Peroxides) for diffusing oxygen to the body through the skin. Such embodiments may be applied for the treatment of decompression injuries.

According to some embodiments of the invention, the external layer may be made of silicone, and the inner layer might be composed of non woven material. The external layer may hold a reservoir of the therapeutic solution.

The external layer may be warmed or cooled as part of the treatment. A Peltier cooler/heater with a temperature regulator may be utilized for this purpose. The inner layer may comprise the therapeutic solution when produced, or be soaked with the therapeutic solution by the user. The inner layer may be comprised of two layers—an inner carrier layer for contacting the skin and wound and a middle layer containing a therapeutic solution.

According to some embodiments of the invention, the medical device may be used for treating any of the following diseases and lesions: skin ulcers, particularly diabetic skin ulcers, skin burns of grades 1, 2 or 3, frost bites, psoriasis, hypertrophic skin disease, edemas, circulatory deficiency, pressure sores, sport injuries, articulation trauma, decompression injuries, hemorrhoids. According to some embodiments of the invention, the medical device may be utilized to cool or to warm a body part.

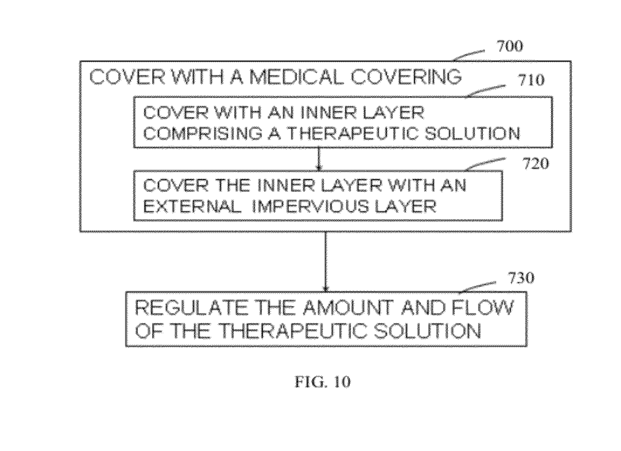

FIG. 10 is a flowchart illustrating a method for healing wounds and skin diseases, according to some embodiments of the invention. The method comprises of the steps: (1) covering an infected skin area or organ with a medical device comprising a therapeutic solution (step 700) and (2) regulating the amount and flow of the therapeutic solution within the medical device (step 730), such that the infected skin area is maintained in contact with the therapeutic solution and leakage is prevented.

It should be pointed out that the step of covering with a medical device (step 700) may comprise of two steps: (1a) covering the infected skin area or organ with an inner layer comprising a therapeutic solution (step 710), and (1b) covering the inner layer with an external impervious and flexible layer (step 720).

It should be further pointed out that the step of covering with an inner layer (step 710) may further comprise: (1ai) covering an infected skin area or organ with a non woven inner layer, and (1aii) covering the non woven inner layer with a therapeutic solution layer. Activating the therapeutic solution layer may take place after the covering of the inner layer with the external layer.

According to some embodiments of the invention, the method may be used utilizing a hypertonic solution comprising at least one of the following: salt, sugar, honey; as the therapeutic solution. According to some embodiments of the invention, the method may be used utilizing at least one of the following: a general antiseptic solution, an antibiotic solution, an antifungal solution, an antiviral solution; as the therapeutic solution.

According to some embodiments of the invention, the method may be used for treating any of the following diseases and lesions: skin ulcers, particularly diabetic skin ulcers, skin burns of grades 1, 2 or 3, frost bites, psoriasis, hypertrophic skin disease, edemas, circulatory deficiency, pressure sores, sport injuries, articulation trauma, decompression injuries, hemorrhoids. According to some embodiments of the invention, the method may be utilized to cool or to warm a body part.

According to another embodiment of the present invention, the hypertonic solution can be added with general antiseptic or antiBiotic or antiFungal or antiViral solutions which come in close proximity with the surface of the infected skin, by the same method of circulating or soaking provided by the present invention.

Each of the layers, namely the outer or inner layer can be used separately, meaning the inner-non woven fabric soaked with medical hypertonic solution can be put in direct contact of the infected skin, such as Face musk or body roll.

The outer layer can be a container/reservoir for hypertonic or medical solutions that can be also wormed or cooled as needed and be used by its own.

According to some embodiments of the invention, the hypertonic solution is either a concentrated sugar solution, such as honey, or a solution of salts. The external layer may be made of silicone, and the inner layer may be made of non woven material. The inner layer may be further comprised of two layers—an inner carrier layer for contacting the skin and wound and a middle layer containing a hypertonic solution. It is well in the scope of the invention, wherein the aforesaid hypertonic solution further comprises effective amount of additives.

It is also in the scope of the invention, wherein the aforesaid hypertonic solution is pre-treated or post-treated. Hence for example, it is one embodiment of the invention, the solution is recycled, enriched, reactivated, dried, sterilized, cleans, filtered, dialyzed, applied by a reverse osmosis, extracted, distillated, purified purged or otherwise cleansed or any combination thereof.

It is also in the scope of the invention, wherein the aforesaid hypertonic solution is recycled or treated in a feed backed manner, e.g., as a function to manual-operated feed backing mechanism, as a function of remote-control communicated interface, as a function of physiological and medical parameters of any combination thereof.

It is also in the scope of the invention, wherein the aforesaid hypertonic solution is treated while being recycled. Hence for example, it is another embodiment of the invention; the solution is recycled while, before or after cooling of heating of the solution. Other operations are possible, e.g., applying ultrasonic vibrations, applying a protocol of treatment combining various sessions, e.g., cooling, US vibrations, drying, applying medicaments, etc.

It is also in the scope of the invention, wherein the aforesaid hypertonic solution comprises at least one carrier.

It is also in the scope of the invention, wherein the aforesaid hypertonic solution comprising effective amount of salts, sugars and honey, hydrogels, polymers, their derivatives or the like.

According an embodiment of the invention, sugars (i.e., any monosaccharides, disaccharides or complex carbohydrates) are selected in a non-limiting manner from one or more compositions selected from a group consisting, inter alia, monosaccharides comprising aldoses and ketoses having a number of about 3 to about carbon atoms per molecule, such as triose (e.g., aldotriose such as glyceraldehyde or ketotriose such as dihydroxyacetone); tetraose (e.g., erythrose and threose, or; pentaose (e.g., arabinose, lyxose, ribose and xylose or ribulose and; hexaose (e.g., allose, altrose, galactose, glucose (i.e., dextrose), gulose, idose, mannose and talose, or fructose, psicose, sorbose and tagatose) etc. A combination of all the sugars is well in the scope of the invention.

Examples of disaccharides are provided in a non-limiting manner: sucrose, lactose, maltose, trehalose, cellobiose etc. Oligosaccharides are saccharide polymers containing a small number (e.g., three to ten) of component sugars, also known as simple sugars, e.g., Fructo-oligosaccharides etc.

Polysaccharides are selected from homopolysaccharides and heteropolysaccharides, especially those selected from a group consisting, inter alia, of alginates, carrageenan, chitin, ficoll, fructans, galactans, glucans, glycosaminoglycans, mannans, pectins, pentosan sulfuric polyester, plant Gums, bacterial polysaccharides, proteoglycans, sepharose, xylans etc.

According to one embodiment of the invention, the hypertonic solution comprises one or more active substances, such as sugars, salts or honey in a range of about 5% to about 95%; or alternatively in the range of 15% to 80%; or alternatively in the range of 30% to 65% or alternatively about 50% (weight percents).

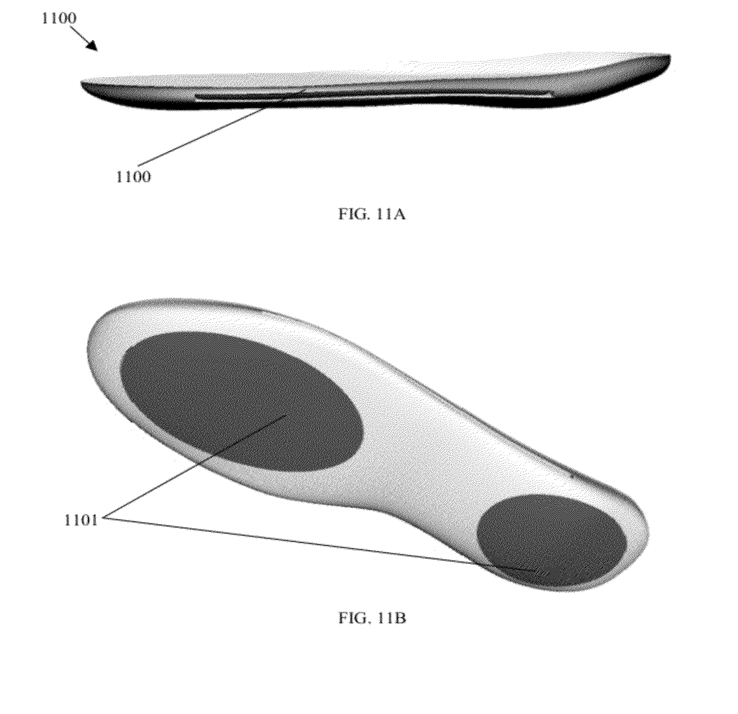

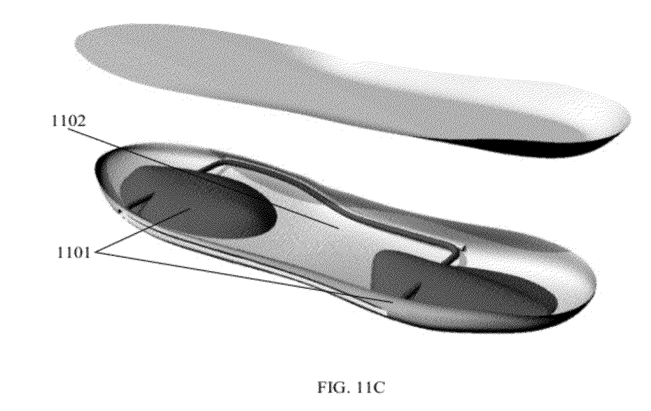

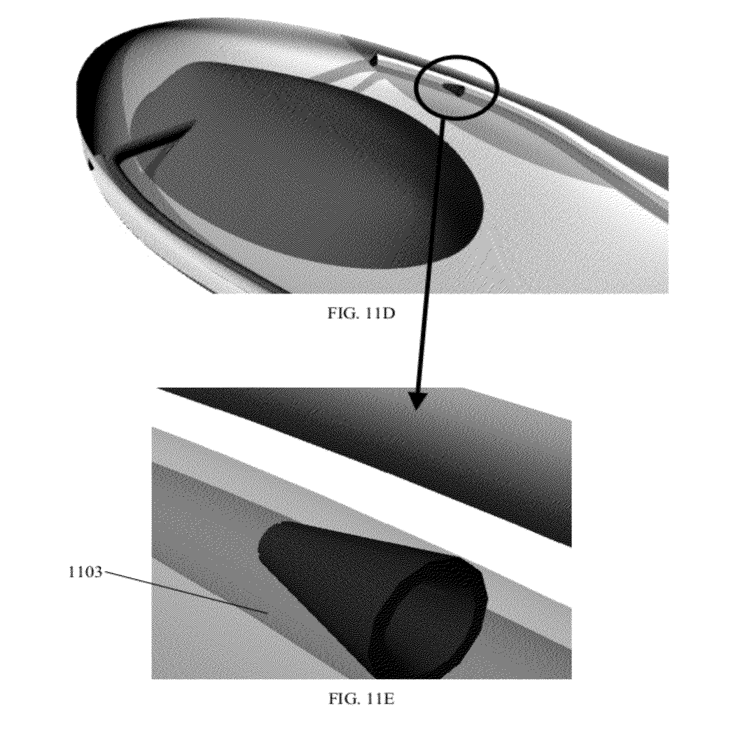

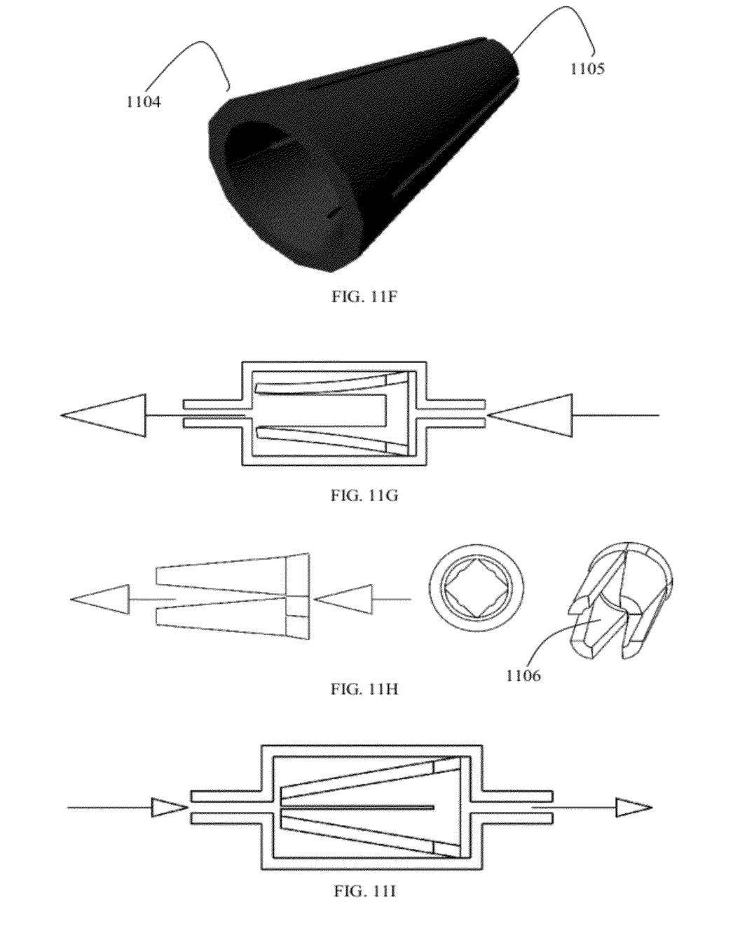

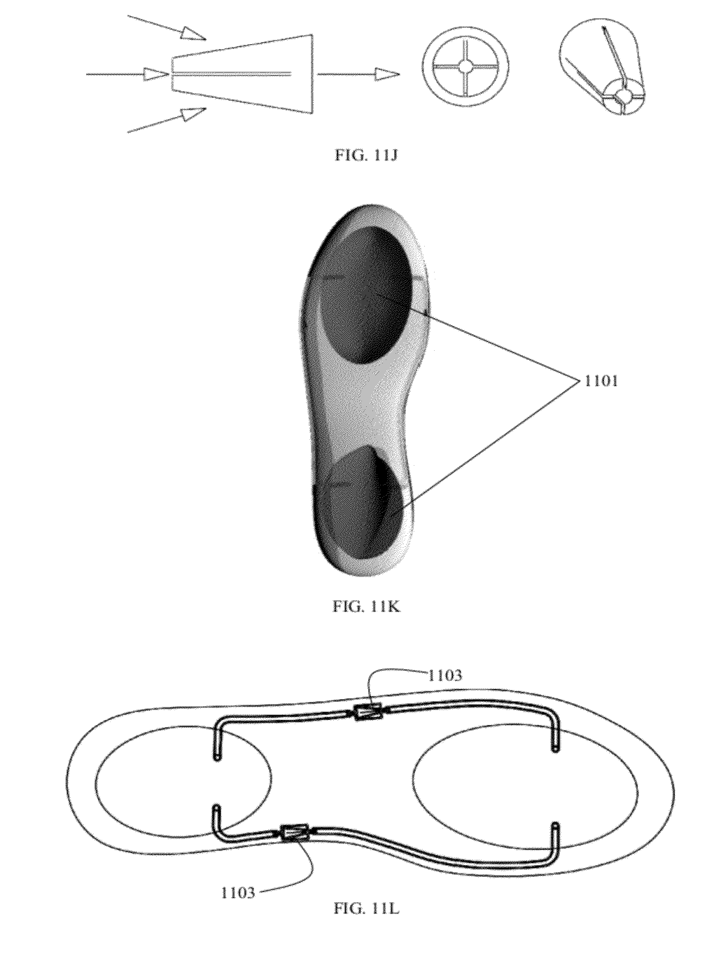

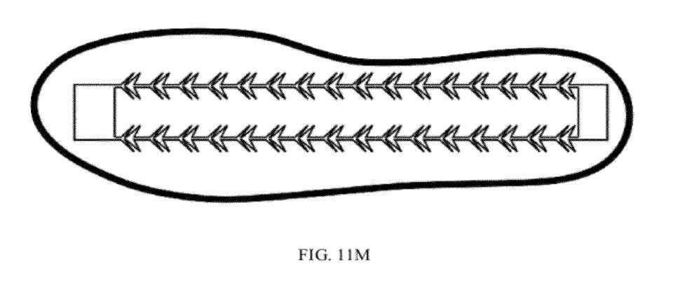

Reference is now made to FIGS. 11A-11M which illustrate another embodiment of the present invention, in which orthopedic foot support 1100 are provided. The orthopedic foot supports 1100 contains at least one water/air reservoir 1101 filled with water/air or another liquid adapted to better scatter the pressure.

The two reservoirs are coupled via at least one lumen 1102, preferably two (as illustrated in FIG. 11C).

Each lumen comprises at least one valve 1103 (see FIG. 11D) so as to enable flow from at least one reservoir to the other. A closer view of the valve is given in FIG. 11E.

It should be pointed out that the valve 1103 may enable either unidirectional flow of liquid or two directional flow of liquid.

FIGS. 11E-11L provides a closer view of the valves and their mode of operation.

According to one embodiment of the present invention, the valves' mechanism is a mechanical one. According to this embodiment the valve is adapted to infuse liquids into the front sole faster and easier than into the rear sole. In other words the rate of liquid flow to the front sole direction is faster than rate of liquid flow to the rear sole direction. Such mechanism is enables due to the cone shape of the valve. This cone shape is characterized by a first large diameter 1104 and a second relatively small diameter 1105 (see FIG. 11F).

Each valve is positioned in lumen 1102 in such a manner that the small diameter 1105 is facing the front sole and the large diameter is facing the rear sole.

When the foot is treading or stepping on the rear sole, the valve ‘opens’, 1106 and a relatively large amount of liquid is forced (in a relative high rate) into the front sole (see FIGS. 11G, 11H).

Alternatively, when the foot is treading or stepping on the front sole a relatively small amount of liquid is forced (in a relative small rate) into the rear sole (see FIGS. 11I, 11J).

It should be emphasized that the valves can be electronically operated.

Furthermore, it should be pointed out that the valves can be reversibly operated. According to this embodiment the valve is adapted to infuse liquids into the rear sole faster and easier than into the front sole. In other words the rate of liquid flow to the rear sole direction is faster than rate of liquid flow to the front sole direction. This can be achieved by (i) simply inversing the direction of the valves; or by (ii) an alternative production of a circulatory foot wrap in which the direction of fluid or air will flow from side to the other by means of the valve direction.

Reference is now made to FIGS. 11L-11M, illustrating the orthopedic foot support 1100 comprising either one valve 1103 (in each lumen 1102) or a plurality of valves 1103.

According to another embodiment, the water/air reservoir 1101 can be located/positioned in different location of the sole for different uses such as Flat feet (pes planus or fallen arches), can be used for disabled or crippled, or simply for pressure relaxation. According to this embodiment, the valves (1103) can be randomly adjusted (in different directions) so as to create/reduce pressure in desired/undesired locations.

According to one embodiment of the invention, the hypertonic solution comprises or admixed with a predetermined volume of gas, such as air, nitrogen, oxygen or an oxygen-enriched fluid for diffusing oxygen to the body through the skin. Such an embodiment may be provided useful in treating decompression injuries.

According to another embodiment, the device as provided by the present invention can be used for non-medical applications such as facial mask, using the orthopedic foot support for better pressure distribution and for absorbing pressure while walking in athletes as well as non-athletes.

It is well within the scope of the invention wherein a disposable member for treating dermatological diseases and topical pathogeneses is disclosed. The disposable member is characterized by (i) size and shape which fit the derma to be treated, (ii) by a matrix at least partially made of non-woven material useful for being continuously wet by a hypertonic solution as defined in any of the above; and (iii) by compatibility with at least one covering external layer facilitating the member to be constantly wet.

According to another embodiment, the disposable member as provided by the present invention comprises means for purification as defined and described in any of the above.

According to another embodiment, the disposable member as provided by the present invention is continuously wet by means of source of hypertonic solution and/or by means of network of pipes as defined and described in any of the above.

While the invention has been described with respect to a limited number of embodiments, these should not be construed as limitations on the scope of the invention, but rather as exemplifications of some of the preferred embodiments. Those skilled in the art will envision other possible variations, modifications, and applications that are also within the scope of the invention. Accordingly, the scope of the invention should not be limited by what has thus far been described, but by the appended claims and their legal equivalents.

1. A medical device for treating dermatological diseases and topical pathogeneses, comprising:
at least one inner layer, at least partially made of a non-woven material, being in physical contact with at least a part of the derma having said dermatological diseases; said inner layer is characterized by being continuously immersed with an effective amount of hypertonic solution such that a constant hypertonic environment around said derma is obtained;
at least one non-disposable external impervious and flexible layer, at least partially made of material selected from a group consisting of silicone, rubber and polymer, enveloping said inner layer;
wherein said inner layer is disposable.

2. The medical device of claim 1, wherein disposable inner layer is adapted to treat dermatological diseases especially de-toxification of said derma and remove pathogeneses by being disposable, frequent displaced and reversibly attached to said enveloping external layer.

3. The medical device of claim 1, wherein said comprising immobilizing means for reversibly tightening said medical device to said derma such that leakage of said hypertonic solution is prevented.

4. The medical device of claim 1, additionally comprising at least one selected from a group consisting of (a) supplying means adapted to provide flow-regulation of said hypertonic solution selected from a group consisting of continuous flow, intervals flow, pulsed flow, programmed flow, pre-programmed flow, flash, circulated or any combination thereof; (b) at least one thermoregulator for thermoregulating the temperature of said solution; or any combination thereof.

5. The medical device of claim 1, wherein said inner layer comprising at least two layers:
a. at least one inner carrier layer being in physical contact with said derma; and,
b. at least one middle layer for temporarily containing said hypertonic solution.

6. The medical device of claim 1, wherein said hypertonic solution is selected from a group consisting of antiseptic solution, a lipophilic solution, an antibiotic solution, an antifungal solution, an antiviral solution or any combination thereof further wherein said hypertonic solution comprising at least one member of a group consisting of: air, oxygen or oxygen-rich liquid, H₂O₂ and peroxides, salt, sugar, honey, 50% dextrose, hypertonic saline, oils, normotonic solutions added with therapeutic aides or any combination thereof.

7. The medical device of claim 1, wherein the configuration of said medical device is selected from a group consisting of a sock-like configuration, glove-like configuration, strap, pre-rolled tube, head-like mask, underwear-like configuration, genital cover, genital wraps, diaper-like, pressure pads, orthopedic foot support.

8. The medical device of claim 1, wherein source of hypertonic solution compromises at least one member of a group consisting of at least one pipe, a network of pipes and of at least one pressure cup for circulating said hypertonic solution within said medical covering.

9. The medical device of claim 1, further comprising purification means, selected from one or more members of a group consisting of beads, fibers, mesh, membrane and filter; further wherein said purification means comprises one member of a group consisting of zinc oxide, black carbon, metal ions, hypochlorite salts, halogen salts, quaternary amine and salts thereof.

10. The medical device of claim 9, wherein said purification means is located in one of the following: (i) within said at least one inner layer, or (ii) in fluid communication with the source of hypertonic solution.

11. A method for treating dermatological diseases and topical pathogeneses comprising steps of:
a. obtaining a device comprising at least one inner layer at least partially made of a non-woven material, being in physical contact with at least a part of the derma having said dermatological diseases; at least one external impervious and flexible layer, at least partially made of material selected from a group consisting of silicone, rubber and polymer, enveloping said inner layer; and a source of hypertonic solution wetting said inner layer such that said inner layer is continuously immersed with said hypertonic solution and a constantly hypertonic environment around said derma is maintained;
b. introducing said device upon at least a part of the derma associated with said dermatological diseases;
c. continuously setting at least one inner layer with said hypertonic solution such that a constant hypertonic environment around said derma is maintained; and
d. frequently replacing said disposable and reversibly detachable at least one inner layer such that de-toxification of said dermal is obtained and topical pathogeneses are removed and dermatological diseases are treated.

12. The method of claim 11, additionally comprising step of providing flow-regulation means of said hypertonic solution; and selecting the same from a group consisting of continuous flow, intervals flow, pulsed flow, programmed flow, pre-programmed flow, flash, circulated or any combination thereof.

13. The method of claim 11, additionally comprising step of thermoregulating the temperature of said solution.

14. The method of claim 11, additionally comprising step of selecting said dermatological diseases from a group consisting of skin ulcers, particularly diabetic skin ulcers, skin burns of grades 1, 2 or 3, frost bites, psoriasis, hypertrophic skin diseases, edemas, circulatory deficiencies, pressure sores, sport injuries, articulation traumas, decompression injuries.

15. The method of claim 11, additionally comprising at least one step selected from (a) composing said hypertonic solution, and selecting the same from one or more member of a group consisting of: salt, sugar, honey, 50% dextrose, hypertonic saline, oils, normotonic solutions added with therapeutic aides or any combination thereof (b) selecting said hypertonic solution from one or more member of a group consisting of antiseptic solution, a lipophilic solution, an antibiotic solution, an antifungal solution, an antiviral solution or any combination thereof.

16. The method of claim 11, additionally comprising steps of configuring said medical device in a configuration; and selecting the same from a group consisting of a sock-like configuration, glove-like configuration, strap, pre-rolled tube, head-like mask, genital cover, genital wraps, diaper-like, pressure pad, orthopedic foot support.

17. The method of claim 11, additionally comprising at least one step selected from (a) regulating the flow of said hypertonic solution; (b) circulating said hypertonic solution whilst homogeneously distributing pressure within said medical device via coupling at least one pressure cup to said external layer; or any combination thereof.

18. The method of claim 11, additionally comprising step of providing said source of hypertonic solution to comprise at least one member of a group consisting of at least one pipe, a network of pipes and of at least one pressure cup for circulating said hypertonic solution within said medical covering.

19. The method of claim 11, additionally comprising at least one step selected from (a) providing means of fluid purification, and (b) selecting said means of fluid purification from one or more members of a group consisting of beads, fibers, mesh, membrane and filter, zinc oxide, black carbon, metal ions, hypochlorite salts, halogen salts, quaternary amine and salts thereof; or any combination thereof.

20. A disposable member for treating dermatological diseases and topical pathogeneses, characterized by (i) size and shape which fit derma to be treated, (ii) by a matrix at least partially made of non-woven medical useful for being continuously wet by a hypertonic solution; and (iii) by compatibility with at least one covering external layer facilitating the member to be constantly wet.
